(19) United States
(12) Patent Application Publication
(10) Pub. No.: US 2016/0220711 A1
(43) Pub. Date: Aug. 4, 2016

(54) METHODS AND COMPOSITIONS FOR TUMOR VASCULATURE IMAGING AND TARGETED THERAPY

(71) Applicant: THE UNIVERSITY OF NORTH CAROLINA AT CHAPEL HILL, Chapel Hill, NC (US)

(72) Inventors: Nancy DeMore, Durham, NC (US); Paul Dayton, Carrboro, NC (US); Russell Mumper, Chapel Hill, NC (US); James K. Tsuruta, Chapel Hill, NC (US)

(21) Appl. No.: 14/917,469

(22) PCT Filed: Sep. 12, 2014
(86) PCT No.: PCT/US2014/055377

§ 371 Date: Mar. 8, 2016

Related U.S. Application Data
(60) Provisional application No. 61/877,526, filed on Sep. 13, 2013.

(51) Int. Cl. A61K 49/22; A61K 41/00; A61K 45/06; A61K 47/48; C07K 16/18

(52) U.S. Cl. CPC A61K 49/221; A61K 49/223; A61K 47/48869; A61K 47/48569; C07K 16/18; A61K 45/06; A61K 41/0028

(57) ABSTRACT

Provided are compositions and methods for imaging one or more of tumor vasculature and tumor angiogenesis, wherein an imaging composition comprises anti-SFRP2 antibody operable linked to an imaging agent. Provided is a method of imaging tumor vasculature in an individual comprising administering to the individual a diagnostically effect of an imaging composition, and detecting the image resulting from the binding or complexing of the imaging composition with tumor vasculature. The imaging agent may also be used as a delivery vehicle to target one or more therapeutic agents to tumor vasculature and surrounding tumor tissue.

### STATEMENT OF PRIORITY

This application claims the benefit of U.S. Provisional Application Ser. No. 61/877,526, filed Sep. 13, 2013, the entire contents of which are incorporated by reference herein.

### STATEMENT OF FEDERAL SUPPORT

This invention was made with government support under Grant Nos. EB009066 and CA142657 awarded by the National Institutes of Health. The government has certain rights in the invention.

### FIELD OF THE INVENTION

This invention relates to methods and compositions for selectively targeting tumor vasculature, and which can be used to distinguish between tumor-associated vasculature and vasculature of normal healthy tissues in an individual, or to direct therapeutic agents to tumor-associated vasculature. More particularly, this invention comprises a composition comprising an antibody to Secreted frizzle-related protein 2 (“SFRP2”) operably linked to an imaging agent. When the imaging agent comprises a contrast agent, the composition may be used as a delivery vehicle to deliver therapeutic agents to tumor vasculature and surrounding tissue, or may be used for in vivo imaging of tumor vasculature for one or more of prognostic and monitoring purposes in an individual having a tumor, including but not limited to in an individual to receive, or having received, therapy targeted against tumor vasculature.

### BACKGROUND OF THE INVENTION

In tumors, creation of new blood vessels (angiogenesis) is dysregulated as compared to the tightly regulated process of angiogenesis in wound healing and tissue repair. As a result, tumor angiogenesis leads to the development of an abnormal vascular network, different in shape, organization, structural dynamics, and permeability that alters the tumor microenvironment in ways which enhance tumor promotion, including the ability of the tumor to grow, progress, metastasize, resist or reduce efficacy of radiotherapy and chemotherapy, and suppress or evade an individual's immune response. Because there are unique features of and factors associated with tumor vasculature, compared with that of normal (healthy and/or noncancerous) tissues, there is commercial and medical interest in selectively targeting tumor vasculature in therapeutic intervention of cancer. The premise is that by selectively targeting tumor vasculature, particularly targeting one or more of the features and factors which enhance tumor promotion, the tumor could be reduced or eliminated.

Selectively targeting tumor vasculature has resulted in at least two different therapeutic approaches, including anti-angiogenic therapy and tumor vascular-disruption therapy. In general, anti-angiogenic therapy is administered to inhibit neovascularization, and induce normalization of the tumor vasculature (e.g., restore regulation of vascularization). The results of anti-angiogenic therapy include prevention or inhibition of tumor growth, disease stabilization, and improvement in response to radiotherapy and chemotherapy. In tumor vascular-disruption therapy, the agents (“tumor vascular-disrupting agents”, or “TVDA”) disrupt established tumor vasculature by one or more processes including, but not limited to, direct apoptotic effects on tumor vasculature endothelial cells, or altering the tubulin cytoskeleton of tumor vasculature endothelial cells thereby inducing shape changes in the endothelial cells, that lead to collapse of existing tumor vasculature, tumor cell death, and tumor necrosis.

Developing along with the selective targeting of therapeutics to tumor vasculature is a need to measure the response in an individual after undergoing such therapy. There is a need for improved compositions and methods for selectively visualizing tumor angiogenesis and/or imaging of tumor vasculature, in particular for measuring or monitoring the effects of therapy targeted against tumor vasculature. There is still a need to provide non-invasive means for imaging the therapeutic efficacy of agents that target tumor vasculature. The degree of therapeutic efficacy detected can then be used in the prognosis of the treated individual, as well as provide information for a clinician to consider with respect to further treatment regimens or modalities for the individual.

Additionally, the extent of tumor vasculature or tumor angiogenesis, such as measured by tumor microvessel density, can be of prognostic value such as for one or more of patient survival time, metastasis, and/or for tumor recurrence after surgical resection. Multiple studies have demonstrated prognostic significance of tumor microvessel density (high density being an adverse prognostic factor) in individuals with gastric cancer, esophageal carcinoma, colorectal carcinoma, pancreatic carcinoma, hepatocellular carcinoma, and breast carcinoma. Importantly, some of these studies also concluded that the prognostic effects of tumor angiogenesis and of biomarker expression (such as HER2, VEGF, etc.) were mutually independent, suggesting the potential benefit of concurrently measuring the extent of tumor angiogenesis by imaging, and biomarker expression, in a prognosis determination. Thus, there is a need for improved compositions and methods for selectively visualizing tumor angiogenesis and/or imaging of tumor vasculature to generate information of prognostic significance in an individual prior to antitumor treatment (e.g., prior to treatment by one or more of chemical, radioisotopic, immunological, surgical, and the like, treatment) or after antitumor treatment.

In addition, there is a need to produce a targeted therapeutic effect on tumor vasculature and surrounding tumor tissue. In that regard, many current anti-tumoral therapeutics have toxicity to healthy tissue as well, so it is desirable to target treatment specifically to the site of the tumor, and reduce systemic effects. A therapeutic agent which can be targeted directly to tumor vasculature, and therefore have a site-specific effect, may improve quality of treatment.

### SUMMARY OF THE INVENTION

The present invention addresses the technical problems and the needs for selectively targeting tumor angiogenesis associated with tumor vasculature by providing methods and compositions that can differentiate between tumor vasculature; and normal vasculature (such as exhibited in healthy tissue); and applying these methods and compositions: (a) for in vivo imaging of tumor vasculature; (b) in a prognostic determination related to an individual having cancer; (c) imaging of tumor vasculature for monitoring the therapeutic efficacy of therapeutic agents directed to tumor vasculature in an individual; (d) in drug development studies, such as preclinical studies in a standard animal xenograft model for tumor, in an assessment whether or not a therapy selectively targets tumor vasculature in vivo and if so, whether the targeting has a therapeutic effect on the tumor vasculature targeted; and (e) as a therapeutic composition by using a therapeutically effective amount of anti-SFRP2 antibody-targeted contrast agent containing one or more therapeutic agents to bind tumor vasculature in vivo, and then exciting the bound, targeted contrast agent with high energy ultrasound sufficient to effect ablation of the tumor vasculature; or as a therapeutic composition by using a therapeutically effective amount of anti-SFRP2 antibody-targeted contrast agent to bind tumor vasculature in vivo, wherein the targeted contrast agent contains one or more chemotherapeutic agents which are delivered to the tumor vasculature and, upon burst, then effect tumor cell death. Also provided are kits for such compositions.

The present invention additionally pertains to methods of performing imaging of tumor vasculature in an individual in need thereof, using an imaging composition comprised of a targeting agent operably linked to an imaging agent; and the imaging composition may optionally further comprise a physiologically acceptable carrier. The targeting agent is anti-SFRP2 antibody, and the imaging agent is an acoustically active vehicle used as a contrast agent for ultrasound imaging, or a fluorescence moiety or label used for optical imaging. In performing the imaging, the imaging composition is contacted with the tumor vasculature, and is detected by a detector capable of detecting the imaging agent. A tumor image can be generated by using a computer and software known in the art to detect and quantify and process signal intensity of the imaging agent, and to generate an image therefrom. In such methods of imaging of tumor vasculature, the imaging composition may be delivered in vivo by administering the imaging composition to an individual; or may be delivered ex vivo, such as by administering the imaging composition to a tissue sample obtained by biopsy of the tumor. In one aspect of the methods of the present invention, assessed is the presence or absence of detection of the imaging composition, wherein detecting the presence of the imaging composition is indicative of the presence of tumor vasculature. In one aspect, detecting the presence of the imaging composition involves detecting and quantifying the amount of imaging composition at the tumor vasculature, as compared to background signal (e.g., non-specific binding of imaging composition to, or autofluorescence of, surrounding tissue other than tumor vasculature), using methods and instruments known in the art, such as by using detectors, computers, and software to process the signals and differentiate signal from imaging composition selectively targeted to tumor vasculature from background signal.

Previously described, by the assignee of the present invention, was a method for detecting angiogenesis in a sample of tissue removed from an individual by measuring the expression or activity of SFRP2 relative to that of a control sample, where an increase in expression or activity is indicative of angiogenesis. Also disclosed was a murine monoclonal antibody that demonstrated the ability to inhibit tumor growth in a murine xenograft model for angiosarcoma and breast carcinoma (see, e.g., PCT Application Publication Number WO 2011/119524 A1). However, application of a humanized antibody to SFRP2 for in vivo imaging of tumor vasculature requires overcoming difficulties to design an antibody-imaging agent composition to have a high target to background ratio, achieve high local concentrations in tumor vasculature to enable imaging of tumor vasculature; and maintain a specificity and selectivity for differentiating between tumor vasculature and vasculature of normal healthy tissue. Such design and composition has been achieved, as illustrated in the accompanying description of invention.

In another aspect of the present invention, methods for treating an angiogenic-dependent disease in an individual in need of such treatment are provided comprising administering to the individual a therapeutically effective amount of a composition comprising anti-SFRP2 antibody-targeted contrast agents comprising one or more therapeutic agents under conditions suitable to promote binding of the anti-SFRP2 antibody-targeted contrast agents to the vasculature expressing SFRP2. The methods and compositions further comprise exciting the targeted contrast agent with high energy ultrasound sufficient for one or more of a) mediating thermal or mechanical effects on the tumor vasculature which cause vascular disruption or collapse, thus having a therapeutic effect; or b) releasing chemotherapeutic agents which are delivered to the tumor vasculature and surrounding tumor tissue, in effecting tumor cell death. The contrast agent serves as a delivery vehicle for one or more therapeutic agents which are in an amount sufficient to effect one or more of anti-angiogenic therapy, vascular-disruption therapy, or antitumor therapy.

### BRIEF DESCRIPTION OF THE DRAWINGS

The foregoing and other features, aspects, and advantages of the invention will be apparent from the following description of the invention as well as the accompanying figures.

FIG. 1 is a bar graph of imaging results plotted by pixel intensity showing that anti-SFRP2 antibody-targeted contrast agent bound specifically to vasculature within tumor. The average pixel intensity observed for anti-SFRP2 antibody-targeted imaging was significantly higher than observed for the streptavidin control microbubbles (“SA”).

FIG. 2 is a graph showing imaging video intensity from anti-SFRP2 targeted microbubble contrast agent correlated significantly with SVR angiosarcoma tumor volume. The baseline-subtracted average pixel intensity for each tumor was plotted against tumor volumes determined using three-dimensional B-mode scans.

FIG. 3 is a bar graph of imaging results plotted by pixel intensity showing that humanized anti-SFRP2 antibody-targeted microbubbles (“Hu-mAb-SFRP2”) bound specifically to vasculature within tumor. The average pixel intensity observed for anti-SFRP2 antibody-targeted imaging was significantly higher than observed for the polyclonal anti-chicken IgY control microbubbles (“Control 2”).

### DETAILED DESCRIPTION OF THE INVENTION

The invention is based on the discovery that an anti-SFRP2 antibody can be used to deliver an imaging agent to selectively target tumor vasculature, in allowing differentiation between tumor vasculature and normal vasculature; thereby providing for acquiring an image of tumor angiogenesis and imaging of tumor vasculature. When the imaging agent comprises a contrast agent, the contrast agent may be used as a delivery vehicle to deliver one or more therapeutic agents preferentially to the tumor vasculature.

While the following terms are believed to be well understood by one of ordinary skill in the art of biotechnology, the following definitions are set forth to facilitate explanation of the invention.

The term “angiogenic-dependent disease” is used herein to refer to a disease characterized by excessive angiogenesis, as compared to healthy tissue of the same tissue origin or type, which occurs when diseased cells produce abnormal amounts of angiogenic growth factors, overwhelming the effects of natural angiogenesis inhibitors. Examples of angiogenic-dependent disease include, but are not limited to, cancer, hemangiomas, fibrosis, diabetic retinopathy, diabetic blindness, age-related macular degeneration, rheumatoid arthritis, psoriasis, uterine fibroids, endometriosis, and dysfunctional uterine bleeding.

The term “antibody” is used herein to refer to an antibody that specifically binds to SFRP2, and includes monoclonal antibodies, polyclonal antibodies, antibody fragments having antigen-binding activity (“antibody fragment”), engineered antibodies (e.g., humanized, for high yield production, for desired pharmacological properties, etc.), chimeric antibodies, and recombinantly produced antibodies. In some embodiments, the antibody is a non-naturally occurring antibody. An “intact antibody” refers to an antibody having two light (L) chains and two heavy (H) chains.

Antigen binding fragments includes Fab, F(ab′)2, Fv, scFv, Fd, and dAB, as known to those skilled in the art. For example, methods are known in the art for generating an Fab fragment, such as deriving it from an antibody by isolating or combining the VH-CR1 domain and VL-CL domain covalently linked by a disulfide bond between the constant region (C). Methods are known in the art for generating a F(ab′)2 fragment comprised of a bivalent fragment having two Fab fragments linked by a disulfide bond at the hinge region. Methods are known in the art for generating an Fv fragment which comprises a VH domain noncovalently linked to a VL domain. Methods are known in the art for generating a single chain Fv (scFv) fragment which comprises either the C-terminus of VH domain linked to the N-terminus of VL domain, or the C-terminus of the VL domain linked to the N-terminus of the VH domain. Methods are known in the art for generating an Fd fragment having two VH and CH1 domains.

Engineered antibodies can be produced by methods known in the art such as by the introduction of conservative amino acid substitutions, consensus sequence substitutions, germline substitutions, deletion of T-cell epitopes, and changes (substitution and/or deletion) in amino acid sequence for altering glycosylation pattern. For example, engineering an antibody to reduce the number of the N-acetylglucosamine residues may be beneficial for promoting half-life, as antibodies with exposed N-acetylglucosamine residues have been shown to be cleared though the mannose receptor and to have a shorter half-life than IgG Fc. In another example, detecting potential T-cell epitopes in an antibody sequence can be performed using commercially available computer modeling software, and the detected epitopes may be eliminated by single or small number amino acid substitution. An antibody may be engineered to achieve one or more of optimization of binding activity, optimization of pharmacodynamic properties, decreasing the immunogenic potential, and optimizing yield in antibody production. Recombinantly produced antibodies can be made by several techniques known in the art to include, but are not limited to, screening protein expression libraries (e.g., phage or ribosomal display libraries). Chimeric antibodies are produced by using methods known in the art (e.g., recombinant DNA techniques) for producing an antibody that has antibody domains obtained from a non-human animal antibody with antibody domains obtained from a human antibody. To determine if an engineered antibody or recombinant antibody or chimeric antibody is operative for the compositions and methods of the invention, a first step is to see if such antibody binds to SFRP2, as binding to SFRP2, and selectively targeting tumor vasculature in which SFRP2 is overexpressed, is an important feature of the invention.

In one example, an epitope is used to immunize an individual to generate an antibody having binding specificity for SFRP2 (“anti-SFRP2 antibody”). Suitable epitopes of human SFRP2 for raising antibodies include, but are not limited to, sequences (numbering based on the GenBank listing for human SFRP2 (accession number AAH08666; SEQ ID NO:17), herein incorporated by reference) comprising, consisting essentially of, or consisting of amino acids 29-40 (GQPDFSYRSNC (SEQ ID NO:1)), 85-96 (KQCHPDTKKELC (SEQ ID NO:2)), 119-125 (VQVKDRC (SEQ ID NO:3)) 138-152 (DMLECDRFPQDNDLC (SEQ ID NO:4)), 173-190 (EACKNKNDDDNDIMETLC (SEQ ID NO:5)), 202-220 (EITYINRDTKIILET KSKT-Cys (SEQ ID NO:6)), or 270-295 (ITSVKRWQKGQREFKRISRSIRKLQC (SEQ ID NO:7)) or a portion thereof of 7 or more contiguous amino acids (e.g., 7, 8, 9, or 10 or more amino acids). In one embodiment, the epitope is amino acids 202-220 (EITYINRDTKIILETKSKT-Cys (SEQ ID NO:6)) or a portion thereof. In another embodiment, the epitope is a sequence of SFRP2 of from about amino acid 156 to about amino acid 295.

In another example, the antibody is a monoclonal antibody produced by hybridoma cell line UNC 68-80 (subclone 80.8.6) (ATCC Deposit No. PTA-11762) which is humanized using methods known in the art, or a humanized antibody that competes for binding, or specifically binds, to the same epitope (SEQ ID NO:6) specifically bound by the monoclonal antibody produced by hybridoma cell line UNC 68-80 (ATCC Deposit No. PTA-11762).

In certain aspects, the monoclonal antibody or a fragment thereof is a chimeric antibody or a humanized antibody. In additional aspects, the chimeric or humanized antibody comprises at least a portion of the CDRs of the monoclonal antibody produced by hybridoma cell line UNC 68-80 (ATCC Deposit No. PTA-11762). As used herein, a “portion” of a CDR is defined as comprising one or more of the three loops from each of the light chain and heavy chain that make up the CDRs (e.g., from 1-6 of the CDRs) which retains the affinity and specificity for binding of SFRP2. In one aspect, the antibody comprises a heavy chain variable region comprising the amino acid sequence of SEQ ID NO:8, or a sequence at least 90% identical thereto, e.g., at least 95, 96, 97, 98, or 99% identical to the amino acid sequence of SEQ ID NO:8 (provided that retained is the affinity and specificity for binding of SFRP2). In one aspect the antibody comprises a light chain variable region comprising the amino acid sequence of SEQ ID NO:9, or a sequence at least 90% identical thereto, e.g., at least 95, 96, 97, 98, or 99% identical to SEQ ID NO:9 (provided that retained is the affinity and specificity for binding of SFRP2). In another aspect, the antibody comprises a heavy chain and light chain selected from the following combinations: a heavy chain variable region comprising the amino acid sequence of SEQ ID NO:8, and a light chain variable region comprising the amino acid sequence of SEQ ID NO:9. For example, humanized anti-SFRP2 antibody for use with the invention can comprise a heavy chain variable region comprising an amino acid sequence of any one of SEQ ID NO:10, SEQ ID NO:11, or SEQ ID NO:12, and a light chain variable region comprising an amino acid sequence of any one of SEQ ID NO:13, SEQ ID NO:14, SEQ ID NO:15, or SEQ ID NO:16; or a sequence at least 90% identical thereto, e.g., at least 95, 96, 97, 98, or 99% identical to such amino acid sequence (provided that retained is the affinity and specificity for binding of SFRP2).

The term “background signal” is used herein to refer to the frequency and magnitude of a signal being detected (e.g., echo, or fluorescence) emitted by a tissue or sample of tissue upon being exposed to an external source used for excitation (e.g., ultrasound; or excitation wavelength in the case of fluorescence) in the absence of administration or binding of the imaging composition of the invention, as distinguished from the signal emitted following the administration and binding of the imaging composition of the invention and exposure to an external source for excitation.

The terms “cancer” or “tumor” are used interchangeably herein to refer to any nonlymphoid tumor. Nonlymphoid tumors are known to include, but are not limited to, angiosarcoma, bladder cancer, breast cancer, cervical cancer, colon cancer, esophageal cancer, gastric cancer, glioblastoma, head and neck cancer, hepatocellular cancer, lung cancer, meningioma, neuroblastoma, ovarian cancer, pancreatic cancer, prostate cancer, rectal cancer, renal cancer, sarcoma, skin cancer, testicular cancer, thyroid cancer, and uterine or cervical cancer.

The term “contrast agent” is used herein to refer to agents which can be detected with increased sensitivity over background signal, either for ultrasound imaging techniques or for optical imaging techniques. In some embodiments, the contrast agent is a non-naturally occurring contrast agent. For ultrasound imaging techniques, contrast agent refers to any microcapsule filled with gas or other material with an acoustic impedance mismatch substantially different from that of tissue and blood, which makes it acoustically active (examples—gas-filled microbubbles, liquid perfluorocarbon droplets). This invention describes compositions comprising tumor-targeted contrast agents for ultrasound imaging which combine the anti-SFRP2 antibody with such an ultrasound contrast agent. The most common type of ultrasound contrast agent is a microbubble. Typically, microbubbles have a mean diameter in a range of about 0.8 to 8 micrometers, and are comprised of a shell comprised of one or more of phospholipid, lipid, albumin, polymer, surfactant, or galactose. The shell encapsulates a gas, including but not limited to gas selected from the group consisting of air, perfluorocarbon (e.g., perfluoropropane, decafluorobutane, etc.), sulfur hexafluoride, and nitrogen. The gas core oscillates when gas-filled microbubbles are caught in an ultrasonic frequency field (the ultrasonic frequency field comprising a source of “excitation”), and reflect a characteristic echo. The echogenicity between the gas in the contrast agent as compared to the surrounding tissue is significantly different, thereby enhancing the reflection of the ultrasound waves to produce a unique image with increased contrast due to the significant difference in echogenicity. Ultrasound imaging may be performed using one or more conventional techniques including, but not limited to, linear contrast-enhanced ultrasound imaging, and nonlinear contrast-enhanced ultrasound imaging, and using methods well known in the art.

For optical imaging techniques, the term “contrast agent” refers to any microsphere, particle, or molecule which can be used in optical fluorescence imaging. This invention describes compositions comprising tumor-targeted optical imaging contrast agents which combine the anti-SFRP2 antibody with such an imaging contrast agent. Ideally, the imaging contrast agent would have an excitation wavelength and emission wavelength in the near-infrared spectrum due to low tissue autofluorescence in this spectrum as well as deep tissue penetration. Preferably the imaging contrast agent has an excitation wavelength in a range of from about 580 nm to 900 nm, and more preferably from about 850 nm to about 800 nm. Various near-infrared fluorescent molecules are commercially available, and include, but are not limited to, rhodamine dyes (Alexa Fluor dyes 660, 680, 700, 750, 790; Texas red), cyanine fluorophores (e.g., Cy5, Cy5.5, Cy7, indocyanine green; pentamethine carbocyanine dyes such as IRD 680, 700, 750, or 800), and quantum dots (semiconductor nanocrystals; e.g., Cu-doped InP/ZnSe, CuInSe, CuInS2/ZnS, CdTe, CdTe/CdSe). Many of the fluorescent molecules are synthesized with a reactive group for operably linking (such as covalently) the fluorescent molecule to a targeting molecule such as a peptide, protein, or antibody using conventional methods and reagents known in the art. For example, reactive groups are known in the art to include, but are not limited to, NHS ester, maleimide, carboxylate, heterobifunctional crosslinker, and a homobifunctional crosslinker.

Contrast agents comprising microbubbles and other acoustically active agents can be used for mediating an antitumor effect themselves, because they can impart thermal or mechanical effects on surrounding vasculature and tissue when excited with appropriate ultrasound parameters. These effects can include either temporary or permanent changes in vascular permeability, vascular disruption, or thermal ablation. Also, the contrast agent may be used as a delivery vehicle for one or more therapeutic agents, Thus, a composition of the invention comprises anti-SFRP2 antibody with an imaging agent, wherein the imaging agent is a contrast agent comprising a delivery vehicle (e.g., microbubble) containing one or more therapeutic agents, which can deliver therapy directly to the tumor site. In that regard, microbubbles and other acoustically active vehicles can also be used as drug carrier or delivery vehicle, where they are loaded with genetic material, or with anti-angiogenic, vascular-disrupting, or antitumor compositions, and then burst at the target site with appropriate ultrasound parameters.

The term “diagnostically effective amount” is used herein to refer to an amount of an imaging composition according to the invention which, when used in a method of imaging or with imaging apparatus, is sufficient to achieve the desired effect of concentrating the imaging agent for imaging one or more of tumor vasculature and tumor angiogenesis in an individual as sought by a researcher or clinician. The amount of an imaging composition of the invention which constitutes a diagnostically effective amount will vary depending on such factors as the contrast agent used, the specificity of the anti-SFRP2 antibody used, the imaging method and apparatus used for imaging, the route of administration, the time of administration, the rate of excretion of the imaging composition, the duration of administration, and the age, body weight, and other health factors of the individual receiving the imaging composition. Such a diagnostically effective amount can be determined routinely by one of ordinary skill in the art having regard to their own knowledge, methods known in the art, and this disclosure.

The term “high energy ultrasound” is used herein to refer to an oscillating sound pressure wave with a peak negative acoustic pressure greater than 800 kiloPascals.

The term “imaging” is used herein to refer to any method or process used to create images or visualization of the tumor vasculature of or from an individual. Typically, imaging is performed in viva, wherein a suitable scanning or imaging technology is used to detect an anti-SFRP2 antibody comprising an imaging agent to selectively target tumor vasculature, in allowing differentiation between tumor vasculature and normal vasculature; thereby providing for visualization of tumor angiogenesis and imaging of tumor vasculature. Any suitable imaging system allowing the detection of a contrast agent (e.g., by ultrasound), or detection of fluorescence-labeled structures (e.g., tumor vasculature), can be applied to the methods, compositions, uses, and kits of the invention. Some imaging systems particularly suitable for in viva imaging of tumor angiogenesis and tumor vasculature are known in the art to include, but are not limited to, the system described in U.S. Published Patent Application US2012/0226119.

The term “imaging agent” is used herein to refer to any compound, composition, or reagent that is detectable for imaging purposes. Imaging agents, include, without limitation, contrast agents and fluorescence-labeled structures. In some embodiments, the imaging agent is a non-naturally occurring imaging agent.

The term “individual” as used herein refers to an animal, a mammal, a human, a non-human primate, a rat, or a mouse.

The term “isolated” as used herein refers to an antibody separated from its natural source from which it was produced (e.g., does not encompass antibody found in blood or tissue which was produced by the individual having the antibody).

The term “kit” is used herein to refer to a combination of reagents, components, and other materials. With respect to the invention, it is contemplated that the kit may include kit components comprising one or more of a targeting agent, an imaging agent, physiologically acceptable carrier, reagents to operably link the targeting agent to the imaging agent in forming the imaging composition of the invention, and the imaging composition; as well as containers for the various components. For example, the targeting agent is an anti-SFRP2 antibody; the imaging agent may be comprised of a contrast agent or a fluorescence moiety or label; and reagents may comprise one more of buffering agents, diluents, and reaction solutions, and molecules (e.g., linker) for conjugating the targeting agent to the imaging agent in forming the imaging composition. It is not intended that the term “kit” be limited to a particular combination of reagents and/or other materials. In one embodiment, the kit further comprises instructions for using the kit components. The kit may be packaged in any suitable manner, typically with the kit components in a single container or various containers as necessary along with a sheet of instructions for carrying out the assay or reaction.

The term “naturally occurring” is used herein to refer to any product or composition that is found in nature.

The term “non-naturally occurring” is used herein to refer to any product or composition that is not found in nature. The term includes compositions in which one or more components is naturally occurring but the combination in the composition is not found in nature.

The term “operably linked” is used herein with respect to the anti-SFRP2 antibody and the imaging agent to refer to a linkage between the anti-SFRP2 antibody and the imaging agent wherein each component retains its activity, i.e., the SFRP2 antibody retains its ability to specifically bind to SFRP2 and the imaging agent retains its ability to provide a signal suitable for imaging.

The term “parenteral” is used herein to refer to administration by injection or infusion, including but not limited to, percutaneous, subcutaneous, intravascular (e.g., intravenous), intramuscular, intrathecal, or intratumoral, and the like.

The term “physiologically acceptable carrier” is used herein to refer to any physiologically compatible medium conventionally used to deliver therapeutics or imaging agents. Such medium may also contain conventional pharmaceutical materials such as, for example, pharmaceutically acceptable salts to adjust the osmotic pressure, buffers, preservatives and the like. Typically, the physiologically acceptable carriers are present in liquid form to facilitate parenteral administration. Illustrative examples of liquids used in carriers include physiological saline, phosphate buffer, normal buffered saline, water, buffered water, 0.4% saline, 0.3% glycine, and may further comprised stabilizers to provide enhanced stability (e.g., glycoproteins, and the like). Since physiologically acceptable carriers are determined in part by the particular route of administration, there are a wide variety of suitable formulations of physiologically acceptable carriers for use with the imaging composition of the present invention. When an imaging composition of the invention is optionally formulated to comprise a physiologically acceptable carrier, a sufficient amount of the anti-SFRP2 antibody operably linked to the imaging agent is present in the physiologically acceptable carrier effective to achieve satisfactory visualization or imaging of the targeted tumor vasculature.

The terms “therapy” and “therapeutics” or “therapeutic agents” when used in reference to targeting vasculature in an angiogenic-dependent disease, are used herein to refer to a therapy or agent that involves contact with vasculature in an angiogenic-dependent disease either directly or via a delivery vehicle, in causing one or more of normalization of vasculature (e.g., restoration of regulation of angiogenesis, or increased oxygenation, or alteration of vessel permeability), inhibition of neovascularization, induction of apoptosis (or other cellular death mechanism) on vasculature endothelial cells in angiogenesis ongoing in the angiogenic-dependent disease, and alteration of tumor vasculature structure or of the cytoskeleton of tumor vasculature endothelial cells. Therapy or therapeutics can include, but are not limited to, anti-angiogenic agents, and tumor vascular-disrupting agents. Tumor vascular-disrupting agents may include, but are not limited to, flavonoids (e.g., vadimezan, flavone acetic acid), xanthenone-4-acetic acid and derivatives (e.g., DMXAA or ASA404), tubulin-binding agents (e.g., crinobulin, fosbretabulin, ombrabulin, plinabulin, soblidotin, dolostatin, AVE8062 (AC-7700), ZD6126 (Angiogene), MPC-6827 (Myriad), 0)(14503 (CA41P-combrestatin A4 phosphate; OxiGene), MN-029 (Medicinova), and BNC105 (Bionomics)). Anti-angiogenic agents can include, but are not limited to, anti-VEGF (vascular endothelial growth factor) agents such as antibodies directed to VEGF or VEGF receptor (e.g., bevacizumab, DC101), small molecules that bind to and inhibit VEGF receptors (e.g., SU6668 (Sugen), TSU68), tyrosine kinase inhibitors of VEGF receptors (e.g., axitinib, sunitnib, sorafenib, and pazopanib), PI3K inhibitor (e.g., PI-103), EGFR inhibitor (gefitinib, erlotinib), Ras inhibitors (FTIs), AKT inhibitor (nelfinavir), anti-SFRP2 antibody, angiostatin, endostatin, and metastatin. When “therapeutics” is used in the context of anticancer therapy, the therapeutics may be selected from one or more chemotherapeutic agents. Examples of chemotherapeutic agents which can be used with or as part of the anti-SFRP2 antibody-targeted microbubbles of the invention include, but are not limited to, DNA methylation inhibitors, LSD1 blockers, PPAR (peroxisome proliferating-activator receptor) ligands (e.g., rosiglitazone); alkylating agents (e.g., nitrogen mustards, such as mechlorethamine, chlorambucil, cyclophosphamide, ifosfamide, and melphalan; nitrosoureas, such as streptozocin, carmustine, and lomustine; alkyl sulfonates, such as busulfan; triazines, such as dacarbazine and temozolomide; ethylenimines, such as thiotepa and altretamine; and platinum-based drugs, such as cisplatin, carboplatin, and oxalaplatin); antimetabolites (e.g., 5-fluorouracil, 6-mercaptopurine, capecitabine, cladribine, clofarabine, cytarabine, floxuridine, fludarabine, gemcitabine, hydroxyurea, methotrexate, pemetrexed, pentostatin, and thioguanine); anti-tumor antibiotics (e.g., anthracyclines, such as daunorubicin, doxorubicin, epirubicin, and idarubicin; and actinomycin-D, bleomycin, mitomycin-C, and mitoxantrone); topoisomerase inhibitors (e.g., topoisomerase I inhibitors such as topotecan and irinotecan; and topoisomerase II inhibitors, such as etoposide, teniposide, and mitoxantrone); mitotic inhibitors (e.g., taxanes, such as paclitaxel and docetaxel; epothilones such as ixabepilone; Vinca alkaloids, such as vinblastine, vincristine, and vinorelbine; and estramustine); corticosteroids (e.g., methylprednisolone, prednisone, and dexamethasone); proteasome inhibitors (e.g., bortezomib); targeted therapies (e.g., imatinib, gefitinib, sunitinib, rituximab, alemtuzumab, trastuzumab, and bortezomib); differentiating agents (e.g., retinoids, tretinoin, and bexarotene); and hormonal agents (e.g., anti-estrogens, such as fulvestrant, tamoxifen, and toremifene); aromatase inhibitors, such as anastrozole, exemestane, and letrozole; progestins, such as megestrol acetate; estrogens; anti-androgens, such as bicalutamide, flutamide, and nilutamide; gonadotropin-releasing hormone (GnRH), also known as luteinizing hormone-releasing hormone (LHRH) agonists or analogs, such as leuprolide and goserelin).

The term “therapeutically effective amount” is used herein to refer to an amount of a composition comprising one or more therapeutic agents according to the invention which, when used in a method of treating an angiogenic-dependent disease, is sufficient to achieve the desired effect of ameliorating, reducing or inhibiting the disease in an individual in need thereof. As known by those skilled in the art such as a clinician, the amount of a composition of the invention which constitutes a therapeutically effective amount will vary depending on such factors as the one or more therapeutic agents used, the specificity of the anti-SFRP2 antibody used, the route of administration, the duration of administration, and the age, body weight, and other health factors of the individual receiving the composition. Such a therapeutically effective amount can be determined routinely by one of ordinary skill in the art having regard to their own knowledge, methods known in the art, and this disclosure.

### EXAMPLES

The following examples are provided to illustrate the practice of the invention, and are not intended to limit the scope of the invention.

### Example 1

This example illustrates various methods of imaging; i.e., using the imaging composition of the invention for one or more of imaging tumor angiogenesis and imaging tumor vasculature. In the various methods of imaging illustrated and within the scope of this invention, the method of detecting the imaging composition bound or complexed to tumor vasculature may comprise distinguishing preferential or specific accumulation of the imaging composition in or on tumor vasculature from background signal. For example, binding of the imaging composition to tumor vasculature is greater than would be expected for accumulation of the imaging composition due to mere circulation or diffusion or the imaging composition such as through normal vasculature or other tissues. In obtaining an image of tumor vasculature or tumor angiogenesis, the method may further or optionally comprise excitation of the imaging agent component of the imaging composition of the invention to generate a signal to be detected. In obtaining an image of tumor vasculature or tumor angiogenesis, the method may further or optionally comprise the signal from the imaging agent component of the imaging composition of the invention being received by a detector for that imaging agent, and the received data is then transmitted to a computer processor. The computer processor can then perform an analysis of the data to determine a result indicating one or more of the presence, amount (e.g., density), and location (within the individual) of tumor vasculature. The process may further or optionally comprise visualizing the results via a visual display unit or printout. The imaging composition may be administered to the individual in a manner where the imaging composition would be expected to encounter tumor vasculature, if present in the individual. Typically, administration parenterally is suitable for this purpose. The imaging agent may comprise a fluorescence moiety or label, or a contrast agent.

### Imaging Prior to Treatment

In one application of the invention, provided are methods and compositions for imaging of tumor vasculature or tumor angiogenesis prior to treatment of an individual having cancer or suspected of having tumor vasculature (e.g., one or more of anti-tumor treatment and treatment targeting tumor vasculature). Imaging prior to treatment can be useful to assess or quantitate the amount of SFRP2 expressed within the tumor microvasculature, which can be of prognostic value such as for one or more of patient survival time; metastasis; for tumor recurrence after surgical resection (in comparing density before and after treatment). A method of imaging tumor vasculature or tumor angiogenesis prior to treatment of an individual having cancer comprises:
(a) administering to the individual a diagnostically effect of an imaging composition comprising an anti-SFRP2 antibody operably linked to an imaging agent, wherein the anti-SRFP2 antibody, when contacted with tumor vasculature, binds or complexes with tumor vasculature;
(b) subsequently detecting the imaging composition bound or complexed to the tumor vasculature, if present; thereby obtaining an image of the tumor vasculature.

### Imaging Post-Treatment (e.g., Following Treatment Comprising Targeting Tumor Vasculature)

In one application of the invention, provided are methods and compositions for imaging of tumor vasculature or tumor angiogenesis subsequent to treatment of an individual having cancer (e.g., one or more of anti-tumor treatment and treatment targeting tumor vasculature). Imaging after treatment can be useful for one or more of monitoring the therapeutic efficacy of therapeutic agents directed to tumor vasculature in an individual; and in drug development studies, such as preclinical studies in a standard animal xenograft model for tumor, in an assessment whether or not a therapy selectively targets tumor vasculature in vivo and if so, whether the targeting has a therapeutic effect on the tumor vasculature targeted. In one aspect, the anti-SFRP2 antibody recognizes and specifically binds to rodent (one or more of mouse and rat) SFRP2 and also recognizes and specifically binds to human SFRP2 (for example, human SFRP2 and mouse SFRP2 have 98% identity). A method of imaging tumor vasculature or tumor angiogenesis in an individual having received treatment for cancer comprises:

(a) administering to the individual a diagnostically effect of an imaging composition comprising an anti-SFRP2 antibody operably linked to an imaging agent, wherein the anti-SRFP2 antibody, when contacted with tumor vasculature, binds or complexes with tumor vasculature;
(b) subsequently detecting the imaging composition bound or complexed to the tumor vasculature, if present; thereby obtaining an image of the tumor vasculature.

### Example 2

In an illustration of making and using an imaging composition according to the invention, a lipid solution containing an 18:1:1 molar ratio of DSPC, PEG2000-PE, PEG2000-PE-Biotin was sonicated to produce lipid encapsulated perfluorobutane microbubbles. Differential centrifugation was used to isolate microbubbles with a mean diameter of approximately 3 microns. Microbubbles were coated with streptavidin by incubating 1×10⁹ microbubbles with 13 μg of streptavidin in PBS. Unbound streptavidin was removed by three sequential washes with PBS, and streptavidin-coated microbubbles were stored at a concentration >1×10⁹ micro-bubbles/ml at 4° C. until needed. The size distribution and concentration of the microbubbles were measured using single particle optical sizing in a commercially available particle size analyzer. Concentrations were reported in particles per ml, and particle diameters were reported in microns. Anti-SFRP2 antibodies were biotinylated using standard procedures known in the art. After combining with streptavidin-coated microbubbles, unbound antibodies were removed by three sequential washes with PBS. The resultant anti-SFRP2 antibody-targeted microbubbles were stored at 4° C. at a concentration >1×10⁹ microbubbles/ml until needed. As an assay control, biotinylated polyclonal antibodies raised in either rabbit or goat against chicken IgY were purchased to serve as a control IgG mixture for the anti-SFRP2 antibodies. The non-targeted control microbubbles were prepared by incubating a (2:1) mixture of the biotinylated goat to biotinylated rabbit antibodies with streptavidin-coated microbubbles, as described above.

Molecular imaging of SFRP2 expression with anti-SFRP2 antibody-targeted microbubbles was performed in an angiosarcoma mouse model. Six week-old male nude mice were injected subcutaneously in their right hind limb with 1×10⁶ SVR angiosarcoma cells. Tumors reached ˜7 mm in length after one week of growth. All ultrasound B-mode images were collected at 15 MHz using a 15L8 linear array transducer with an ultrasound imaging system to provide images for selecting the region of interest in each imaging plane. CPS mode, a nondestructive contrast-specific imaging technique operating at 7 MHz (mechanical index=0.18, CPS gain=−3 dB) was used to image targeted and control microbubbles as contrast agents. Molecular imaging of SFRP2 expression in tumor vasculature was performed with anti-SFRP2 antibody-targeted microbubbles. Briefly, a 3-dimensional (3D) scan of the angiosarcoma tumor was performed in B-mode to record the outline of the tumor. As an assay control, 5×10⁶ streptavidin-coated microbubbles (no antibody attached; “Control 1”) in approximately 50 μl of saline were injected into the tail vein of nude mice with angiosarcoma tumors. The perfusion of the tumor and surrounding tissue by Control 1 microbubbles was captured in Cadence mode. Approximately 18 minutes were required for all free-flowing Control 1 microbubbles to clear from the vasculature. At this point a 3D scan of the tumor and surrounding tissue was recorded in Cadence mode to capture signal from Control 1 microbubbles that remained within the tumor. A baseline 3D scan was acquired after destroying Control 1 microbubbles retained within the tumor with a high-energy D color scan. Anti-SFRP2 antibody-targeted microbubbles (5×10⁶ micro bubbles in ˜50 μl of saline) were used in an identical manner to determine the expression of SFRP2 within the angiosarcoma tumors.

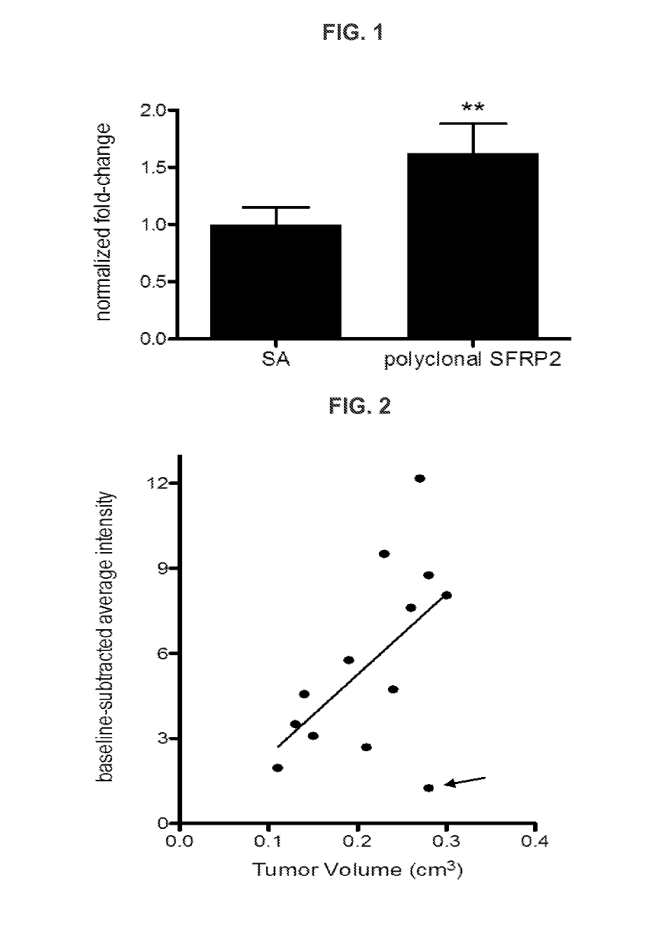

As shown in FIG. 1, anti-SFRP2 antibody-targeted microbubbles detected tumor vasculature with significantly more signal intensity than Control 1 microbubbles (FIG. 1, “SA”). The normalized fold-change was 1.6±0.27 (n=13, p=0.0032). After allowing all freely flowing contrast agent to be cleared from the circulation, anti-SFRP2 antibody-targeted microbubbles were retained only in the vasculature within the borders of the allograft, and surrounding tissue had no significant echogenicity. Likewise, the Control 1 microbubbles were retained within the vasculature within the borders of the allograft, with no significant signal from the surrounding normal tissue.

In a separate experiment, the retention of Streptavidin-coated microbubbles with a 2:1 mixture of biotinylated goat α-chicken IgY and biotinylated rabbit α-chicken IgY (α-chicken IgY-microbubbles) was tested as another assay control (“Control 2”). Compared was the baseline-subtracted average pixel intensity of Control 1 microbubbles to Control 2 microbubbles using an unpaired, two-tailed t-test. Control 2 microbubbles were retained within the tumor vasculature at significantly lower levels than Control 1 microbubbles (p=0.0002). Control 2 microbubbles had an average pixel intensity 5-fold lower than the Control 1 microbubbles. Accordingly, it was calculated that anti-SFRP2 antibody-targeted microbubbles would have average baseline-corrected pixel intensity 8-times higher than Control 2 microbubbles.

Anti-SFRP2 antibody-targeted microbubbles were then analyzed for specificity to tumor vasculature using the angiosarcoma mouse model. Using ultrasound imaging, the signals from Control 1 microbubbles and anti-SFRP2 antibody-targeted microbubbles were apparent throughout the tumor and surrounding normal tissue while these contrast agents were freely circulating through the vasculature. However, after allowing all freely flowing contrast agent to be removed from circulation, video signal was significantly lower in the normal tissue surrounding the tumor than within the tumor. This demonstrated that the Control 1 microbubbles and anti-SFRP2 antibody-targeted microbubbles did not bind significantly within normal vasculature. In addition, examined was the video intensity in the kidney and in the liver. Both the Control 1 microbubbles and anti-SFRP2 antibody-targeted microbubbles were retained within the liver, resulting in intense echogenicity. On the other hand, kidney was largely devoid of echogenicity with no significant difference between the Control 1 microbubbles and anti-SFRP2 antibody-targeted microbubbles. As shown in FIG. 2, when average pixel intensity obtained from anti-SFRP2 antibody-targeted microbubbles against tumor volume was plotted, the general finding was that average pixel intensity increased as tumor volume increased. Only one of thirteen animals in the angiosarcoma mouse model examined had higher average pixel intensity for the Control 1 microbubbles than for anti-SFRP2 antibody-targeted microbubbles (indicated by the arrow in FIG. 2). Correlation analysis showed a highly significant relationship (p=0.003) between tumor volume and video signal from anti-SFRP2 antibody-targeted microbubbles, with value of 0.60 for r² (Pearson) when omitting the aforementioned “outlier” from the Control 1 microbubbles. Even when this later data point was included in the correlation analysis, there was a significant relationship between tumor volume and video signal from anti-SFRP2 antibody-targeted microbubbles (p=0.048) with a value of 0.31 for r² (Pearson) as illustrated by the best-fit line in FIG. 2. In the range of tumor volumes investigated, as tumors increased in volume: either SFRP2 expression increased, or the number of vessels expressing SFRP2 increased, or there was some combination of increased vessel number and SFRP2 expression. These results show that an imaging agent comprising anti-SFRP2 antibody-targeted microbubbles can be used (a) for selectively targeting tumor angiogenesis and for imaging SFRP2, a molecular marker associated with tumor vasculature; and (b) for differentiating between tumor vasculature, and normal vasculature.

### Example 3

In this Example, illustrated are additional methods for making and using an imaging composition according to the invention. Methods and reagents, additional to those described in Example 2 herein, can be used to operably link the anti-SFRP2 antibody to the acoustically active agent. For example, using methods known in the art, a bifunctional linker having a maleimide functionality at one end of the linker, and a hydrazide functionality at the other end of the linker can be used to operably link the anti-SFRP2 antibody to the acoustically active agent. The hydrazide functionality can attach, via hydrazine formation, to oxidized carbohydrates on the antibody. The resultant antibody-linker conjugate is then reacted with acoustically active agent having a thiol functionality available for binding to the maleimide functionality. A polymer, used to create an acoustically active agent, can be functionalized with thiol functionalities prior to mixing with lipid and gas components of the acoustically active agent. In this regard, poly(acrylic acid) (PAA), which binds to phosphocholine headgroups, can be partially functionalized with cysteamine to add thiol groups. The thiol-functionalized polymer can then be combined with a premade suspension of 1,2-distearoyl-sn-glyerco-3-phospho-choline (DSPC) in pH 3.4 acetate buffered saline in forming a suspension. Perfluorobutane gas can then be flowed into the headspace above the suspension, and then the mixture is sonicated at the gas-liquid interface to form acoustically active agent which has a thiol functionality available for binding to a maleimide functionality.

Additionally, a humanized anti-SFRP2 monoclonal antibody was used to create an imaging composition according to the invention. Size-sorted micro-bubble ultrasound contrast agent containing biotin was prepared using differential centrifugation. The biotinylated micro-bubbles were coated with a molar excess of streptavidin. Excess streptavidin from the coated microbubbles was removed by washing with phosphate buffered saline. The carbohydrate chains on a humanized anti-SFRP2 antibody were mildly oxidized with sodium meta-periodate to create reactive carbonyls. Biotin was then added to the humanized antibody by incubating a reagent comprising hydrazide-PEG4-biotin with the reactive carbonyls according to the directions of the reagent manufacturer. Excess biotinylation reagent was removed by gel-filtration with phosphate buffered saline. Used as assay control antibodies were two commercially available biotinylated antibodies (biotinylated rabbit anti-chicken IgY and biotinylated goat anti-chicken IgY). Incubating a molar excess of the biotinylated humanized anti-SFRP2 antibody with the streptavidin-microbubbles created the humanized anti-SFRP2 antibody-targeted microbubbles. The goat anti-chicken IgY was combined with the rabbit anti-chicken IgY in a (2:1) ratio. Adding a molar excess of this control antibody solution to the streptavidin-microbubbles created the assay negative control microbubbles (“Control 2 microbubbles”). In both cases, unbound antibody was removed by washing with PBS. The size distribution and concentration of the final preparation of targeted or control contrast agent was determined using a particle sizing system.

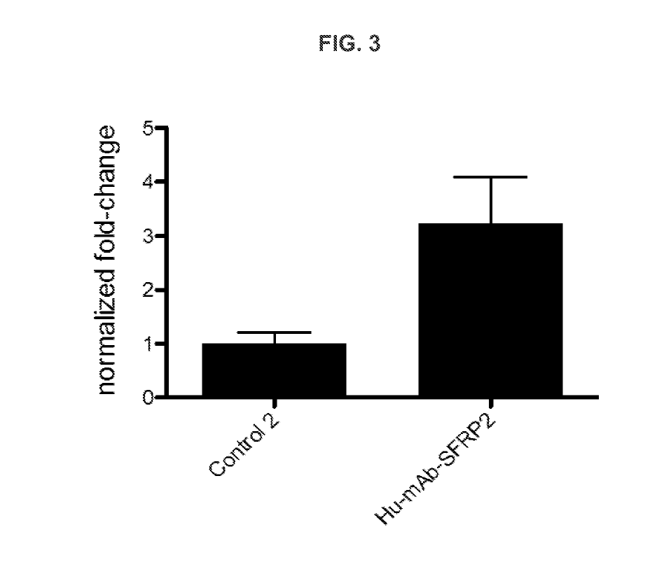

The humanized anti-SFRP2 antibody-targeted microbubbles were then analyzed for specificity to tumor vasculature using the angiosarcoma mouse model, as described above in Example 2. Ultrasound imaging was used to quantitate the uptake in tumor vessels of the Control 2 microbubbles and anti-SFRP2 antibody-targeted microbubbles. As shown by the ultrasound imaging, the Control 2 microbubbles had only minimal uptake in tumor vessels. In contrast, the ultrasound imaging showed that the anti-SFRP2 antibody-targeted microbubbles had significant uptake in tumor vessels. As shown in FIG. 3, humanized anti-SFRP2 antibody-targeted microbubbles (“Hu-mAb-SFRP2”) detected tumor vasculature with significantly more signal intensity than Control 2 microbubbles (“Control 2”). The normalized fold-change was greater than 2 fold (p=0.0108). These results show that an imaging agent comprising anti-SFRP2 antibody-targeted microbubbles can be used (a) for selectively targeting tumor angiogenesis and for imaging SFRP2, a molecular marker associated with tumor vasculature; and (b) for differentiating between tumor vasculature and normal vasculature.

### Example 4

In this Example, illustrated is the use of a composition of the invention comprising anti-SFRP2 antibody-targeted contrast agent comprising microbubbles, and use of a composition wherein anti-SFRP2 antibody-targeted microbubbles further comprise one or more therapeutic agents (or a pharmaceutical composition comprising such composition and a physiologically acceptable carrier) in a therapeutically effective amount to treat an angiogenic-dependent disease in an individual in need of such treatment. In one aspect of the invention, provided is a method for treating an angiogenic-dependent disease, in an individual in need of such treatment, comprising the steps of (a) administering to an individual (a mammal, such as a human) in need of such treatment, a therapeutically effective amount of a composition comprising anti-SFRP2 antibody-targeted microbubbles; (b) allowing sufficient time for the composition to bind to SFRP2 on vasculature present in the individual with angiogenic-dependent disease in sufficient concentrations; and (c) applying ultrasound at an energy sufficient to cause the microbubbles to burst and mediate a therapeutic effect. The therapeutic effect may be one or more of anti-angiogenic therapy and vascular-disruption therapy.

In another aspect, the composition may further comprise one or more therapeutic agents. A contrast agent, used as a delivery vehicle, may be loaded with one or more therapeutic agents using methods known in the art. For example, a therapeutic agent may be encapsulated by the microbubble, entrapped or incorporated in the microbubble, or associated with (e.g., bound to) the microbubble, in any one of a number of ways known to those skilled in the art. For example, a hydrophobic coating can be applied to the therapeutic agent. The coated therapeutic agent is then introduced into a PBS/lipid solution, in the presence of the gas, followed by sonication or vigorous shaking. This can form an acoustic emulsion containing microbubbles which have entrapped therein the hydrophobically coated therapeutic agent. Therapeutic agents may also be entrapped within the membrane shell of a lipid-coated microbubble. Thus, in a method of treating an angiogenic-dependent disease, following burst of the microbubbles, released is the one or more therapeutic agents at the site of the vasculature targeted by anti-SFRP2 antibody-targeted microbubbles. In the case where the vasculature is tumor vasculature and the one or more therapeutic agents comprise one or more chemotherapeutic agents, the one or more chemotherapeutic agents are delivered to the tumor for mediating an antitumor effect.

1. A method of imaging one or more of tumor vasculature or tumor angiogenesis in an individual having cancer, comprising administering to the individual a diagnostically effective amount of an imaging composition comprising an anti-SFRP2 antibody operably linked to an imaging agent, wherein the imaging composition binds or complexes with SFRP2 of tumor vasculature, and detecting the image resulting from the binding or complexing of the imaging composition with tumor vasculature.

2. A method of imaging one or more of tumor vasculature or tumor angiogenesis in an individual comprising:
(a) administering to the individual a diagnostically effect of an imaging composition comprising an anti-SFRP2 antibody operably linked to an imaging agent, wherein the anti-SRFP2 antibody, when contacted with tumor vasculature, binds to or complexes with tumor vasculature;
(b) subsequently detecting the imaging composition bound or complexed to the tumor vasculature, if present; thereby obtaining an image of the tumor vasculature.

3. The method of claim 2, wherein the method is performed for a use selected from the group consisting of in vivo imaging of tumor vessels, prognostically, monitoring the therapeutic efficacy of therapeutic agents directed to tumor vasculature, and in drug development studies.

4. The method of claim 2, wherein the imaging composition further comprises a physiologically acceptable carrier.

5. The method of claim 2, wherein the imaging agent is selected from the group consisting of a fluorescent particle, a fluorescent moiety and a fluorescent label.

6. The method of claim 2, wherein the imaging agent comprises an acoustically active agent.

7. The method of claim 6, wherein the acoustically active agent is selected from the group consisting of a microbubble and a perfluorocarbon droplet.

8. The method of claim 6, wherein the imaging agent comprises a delivery vehicle loaded with one or more therapeutic agents.

9. An imaging composition comprising an anti-SFRP2 antibody operably linked to an imaging agent.

10. The imaging composition of claim 9, further comprising a physiologically acceptable carrier.

11. The imaging composition of claim 9, wherein the imaging agent is selected from the group consisting of a fluorescent particle, a fluorescent moiety and a fluorescent label.

12. The imaging composition of claim 9, wherein the imaging agent comprises an acoustically active agent.

13. The imaging composition of claim 12, wherein the acoustically active agent is selected from the group consisting of a microbubble and a perfluorocarbon droplet.

14. The imaging composition of claim 12, wherein the imaging agent comprises a delivery vehicle loaded with one or more therapeutic agents.

15. A method of delivering therapy to one or more of tumor vasculature or tumor angiogenesis in an individual comprising:
administering to the individual a composition comprising an anti-SFRP2 antibody operably linked to an acoustically active agent, wherein the anti-SRFP2 antibody, when contacted with tumor vasculature, binds to or complexes with tumor vasculature.

16. The method of claim 15, further comprising exciting the acoustically active agent with sufficient acoustic energy to mediate one or more of thermal effects or mechanical effects on one or more of the tumor microvasculature or surrounding tissue.

17. The method of claim 15, where the composition is an acoustically active agent which has been loaded with one or more therapeutic agents.

18. The method of claim 17, further comprising exciting the acoustically active agent with sufficient acoustic energy to release the one or more therapeutic agents from the acoustically active agent.

19. The method of claim 15, wherein the acoustically active agent is selected from the group consisting of a microbubble and a perfluorocarbon droplet.

20. A pharmaceutical composition comprising an anti-SFRP2 antibody operably linked to an acoustically active agent loaded with at least one therapeutic agent.

21. The pharmaceutical composition of claim 20, further comprising a physiologically acceptable carrier.

22. The pharmaceutical composition of claim 20, wherein the at least one therapeutic agent is a chemotherapeutic agent.

23. The pharmaceutical composition of claim 20, wherein the acoustically active agent is selected from the group consisting of a microbubble and a perfluorocarbon droplet.

24. A method for treating an angiogenic-dependent disease, in an individual in need of such treatment, comprising the steps of (a) administering to an individual in need of such treatment a therapeutically effective amount of a composition comprising anti-SFRP2 antibody-targeted acoustically active agent; (b) allowing sufficient time for the composition to bind to SFRP2 on vasculature present in the individual with angiogenic-dependent disease in sufficient concentrations; and (c) applying ultrasound at an energy sufficient to cause the acoustically active agent to burst and mediate a therapeutic effect.

25. The method of claim 24, wherein the composition further comprises one or more therapeutic agents.

26. The method of claim 25, wherein the one or more therapeutic agents comprises a chemotherapeutic agent.

27. The method of claim 24, wherein the acoustically active agent is selected from the group consisting of a microbubble and a perfluorocarbon droplet.


SEQUENCE LISTING (17 sequences)

SEQ ID NO: 1 - protein, 11 aa, Artificial
  Epitope sequence for generating anti-SFRP2 antibodies
GQPDFSYRSNC

SEQ ID NO: 2 - protein, 12 aa, Artificial
  Epitope sequence for generating anti-SFRP2 antibodies
KQCHPDTKKELC

SEQ ID NO: 3 - protein, 7 aa, Artificial
  Epitope sequence for generating anti-SFRP2 antibodies
VQVKDRC

SEQ ID NO: 4 - protein, 15 aa, Artificial
  Epitope sequence for generating anti-SFRP2 antibodies
DMLECDRFPQDNDLC

SEQ ID NO: 5 - protein, 18 aa, Artificial
  Epitope sequence for generating anti-SFRP2 antibodies
EACKNKNDDDNDIMETLC

SEQ ID NO: 6 - protein, 20 aa, Artificial
  Epitope sequence for generating anti-SFRP2 antibodies
EITYINRDTKIILETKSKTC

SEQ ID NO: 7 - protein, 26 aa, Artificial
  Epitope sequence for generating anti-SFRP2 antibodies
ITSVKRWQKGQREFKRISRSIRKLQC

SEQ ID NO: 8 - protein, 120 aa, Artificial
  Anti-SFRP2 antibody heavy chain variable region sequence
QVQLQQPGAELVQPGASVMLSCKASGFTFTRYWWHWVRQTPGRGLEWIGRIDPNSGTTRFIEKFKTKATLTVDKPSSTAYMHLSSLTSEDSAVYYCARWGPYYGYAMDYWGPGTSVTVSS

SEQ ID NO: 9 - protein, 106 aa, Artificial
  Anti-SFRP2 antibody light chain variable region sequence
QIVLTQSPAIMSASPGQKVTITCSASSSVTYMHWYQQKLGSSPKLWIYDTSRLAPGSPARFSGSGSGTSYSLTISSMETEDAASYFCHQWSTYPPTFGTGTKLEIQ

SEQ ID NO: 10 - protein, 120 aa, Artificial
  Anti-SFRP2 antibody heavy chain variable region sequence
QVQLVQSGAELKKPGASVKVSCKASGFTFTRYWWHWVRQAPGKGLEWIGRIDPNSGTTRFIEKFKTRATITVDKSTSTAYMHLSSLRSEDSAVYYCARWGPYYGYAMDYWGQGTSVTVSS

SEQ ID NO: 11 - protein, 120 aa, Artificial
  Anti-SFRP2 antibody heavy chain variable region sequence
QVQLVQSGAEVKKPGASVKVSCKASGFTFTRYWWHWVRQAPGKGLEWIGRIDPNSGTTRFIEKFKTRATITVDKSTSTAYMELSSLRSEDTAVYYCARWGPYYGYAMDYWGQGTLVTVSS

SEQ ID NO: 12 - protein, 120 aa, Artificial
  Anti-SFRP2 antibody heavy chain variable region sequence
QVQLVQSGAEVKKPGASVKVSCKASGFTFTRYWWHWVRQAPGKGLEWIGRIDPNSGTTRFIEKFKTRVTITVDKSTSTAYMELSSLRSEDTAVYYCARWGPYYGYAMDYWGQGTLVTVSS

SEQ ID NO: 13 - protein, 106 aa, Artificial
  Anti-SFRP2 antibody light chain variable region sequence
QIVLTQSPAILSLSPGERVTITCSASSSVTYMHWYQQKLGKAPKLWIYDTSRLAPGSPARFSGSGSGTDYTLTISSLETEDFASYFCHQWSTYPPTFGQGTKLEIK

SEQ ID NO: 14 - protein, 106 aa, Artificial
  Anti-SFRP2 antibody light chain variable region sequence
QIVLTQSPATLSLSPGERVTITCSASSSVTYMHWYQQKLGKAPKLWIYDTSRLAPGSPARFSGSGSGTDYTLTISSLESEDFASYFCHQWSTYPPTFGQGTKLEIK

SEQ ID NO: 15 - protein, 106 aa, Artificial
  Anti-SFRP2 antibody light chain variable region sequence
QIVLTQSPATLSLSPGERVTITCSASSSVTYMHWYQQKPGKAPKLWIYDTSRLAPGSPARFSGSGSGTDYTLTISSLESEDFASYFCHQWSTYPPTFGQGTKLEIK

SEQ ID NO: 16 - protein, 106 aa, Artificial
  Anti-SFRP2 antibody light chain variable region sequence
QIVLTQSPATLSLSPGERVTITCSASSSVTYMHWYQQKPGKAPKLWIYDTSRLAPGSPARFSGSGSGTDYTLTISSLESEDFATYFCHQWSTYPPTFGQGTKLEIK

SEQ ID NO: 17 - protein, 295 aa, Homo sapiens [sequence omitted]